(19) United States
(12) Patent Application Publication
(10) Pub. No.: US 2020/0075147 A1
(43) Pub. Date: Mar. 5, 2020

(54) REMOTE CONTROLLED DIGITAL SYSTEM THAT CONNECTS PATIENTS AND HEALTHCARE PROFESSIONALS

(71) Applicant: Ali Mahmoud FARHAT, Dearborn, MI (US)

(72) Inventor: Ali Mahmoud FARHAT, Dearborn, MI (US)

(21) Appl. No.: 16/554,200

(22) Filed: Aug. 28, 2019

Related U.S. Application Data
(63) Continuation-in-part of application No. 15/958,650, filed on Apr. 20, 2018, now abandoned.
Application No. 15/958,650 is a continuation of application No. 15/599,951, filed on May 19, 2017, now abandoned.
Application No. 15/599,951 is a continuation-in-part of application No. 15/472,344, filed on Mar. 29, 2017, now abandoned.

(51) Int. Cl. G16H 20/10; G16H 15/00; G06Q 20/08; G06Q 30/06; G16H 20/17; G16H 80/00; G06Q 50/22; G06Q 20/40; G06Q 20/32; G06Q 20/10; G16H 40/20

(52) U.S. Cl. CPC G16H 20/10; G16H 15/00; G06Q 20/085; G06Q 30/0603; G06Q 30/0643; G16H 40/20; G16H 80/00; G06Q 50/22; G06Q 20/4014; G06Q 20/322; G06Q 20/102; G16H 20/17

(57) ABSTRACT

A healthcare services provider system for providing an informational processing interface at a mobile device includes a mobile device including an interface configured to (1) present a display screen to receive user input to control a central processing unit that is operably coupled to at least one healthcare provider; (2) transmit, via a wireless network, a mobile device command indicating a scheduling request or inquiry, the command being generated in response to user input, (3) receive, via the wireless network and in response to the mobile device command, first data indicative of the status of the remote central processing unit, and; (4) present an updated display screen, the updated display screen reflecting the responsive information and history if any as a result of the mobile device command to present a real-time display from the remote central processing unit.

### CROSS-REFERENCE TO RELATED APPLICATIONS

This application is a continuation-in-part of U.S. application Ser. No. 15/958,650, filed on Apr. 20, 2018, which is a continuation of U.S. application Ser. No. 15/599,951, filed on May 19, 2017, which is a continuation-in-part of U.S. application Ser. No. 15/472,344 filed on Mar. 29, 2017, the disclosures of which are incorporated in their entireties by reference herein.

### TECHNICAL FIELD

Embodiments disclosed herein generally relate to a mobile application and web-based system that wirelessly connects patients and healthcare professionals.

### SUMMARY

Disclosed is a mobile application and web-based system (collectively, “system”) that connects patients and healthcare professionals. As a result of interactions therebetween, a complete medical “wallet” may be created. In some embodiments, the medical wallet allows the patient to have his or her medical records, a list of medications and physicians on hand for themselves or their loved ones. These may include, for example, pets. The system gives the patient access to healthcare professionals such as physicians, dentists, pharmacies, laboratories and veterinarians in any chosen geographic area nationwide.

Several aspects of the system connect patients to doctors through a secure mobile application. Both the patient and the doctor may access the patient's medical profile, sports forms, medical lab reports and prescription information, among other features. The mobile application and web-based system are HIPAA-compliant and may have bank-rated encryption.

Several features aspects of the system allow the patient to have his/her medical information easily accessible. The system contains the necessary information for patients to locate doctors who take his or her insurance, allows the patient to share his or her medical profile and fill out and share forms. Patients can schedule appointments, securely chat with a participating doctor, receive prescriptions from that doctor and share the prescription(s) with his or her pharmacy.

### BRIEF DESCRIPTION OF THE DRAWINGS

Several embodiments of the present disclosure are pointed out with particularity in the appended claims. However, other features of the various embodiments will become more apparent and will be best understood by referring to the following detailed description in conjunction with the accompanying drawings, in which:

FIG. 1 illustrates the main steps in using one variation of the disclosed system;

FIG. 2 illustrates an example user interface to facilitate capturing information about prescriptions, family members and (optionally) pets;

FIG. 3 illustrates an example user interface in which a patient is invited to add family details;

FIG. 4 illustrates an example user interface in which a patient is invited to add details of prescriptions, the prescribing health professional and prescription duration;

FIG. 5 illustrates an example user interface in which a patient may view details of upcoming and past appointments;

FIG. 6 illustrates an example user interface in which a patient is invited to send and read messages and otherwise interact with a healthcare provider, in this case through a chat room;

FIG. 7 is an example user interface in which details of a patient's account may be entered;

FIG. 8 illustrates an example of user/patient registration (initialization and setup) including interactions between a user, a remote device and a healthcare professional via a central processing unit in accordance with one embodiment;

FIG. 9 illustrates an example transaction between the central processing unit and a healthcare provider in accordance with one embodiment; and

FIG. 10 illustrates an example transaction between the user via the remote device and the central processing unit and a healthcare provider to schedule an appointment and make inquiries in accordance with one embodiment;

### DETAILED DESCRIPTION

As required, detailed embodiments of the present invention are disclosed herein; however, it is to be understood that the disclosed embodiments are merely examples of the invention that may be embodied in various and alternative forms. The Figures are not necessarily to scale; some features may be exaggerated or minimized to show relevant details. Therefore, specific structural and functional details disclosed herein are not to be interpreted as limiting, but merely as a representative basis for teaching one skilled in the art to variously employ the present invention.

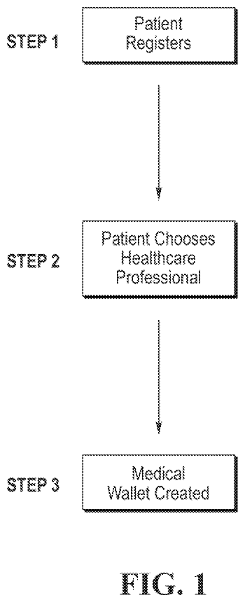

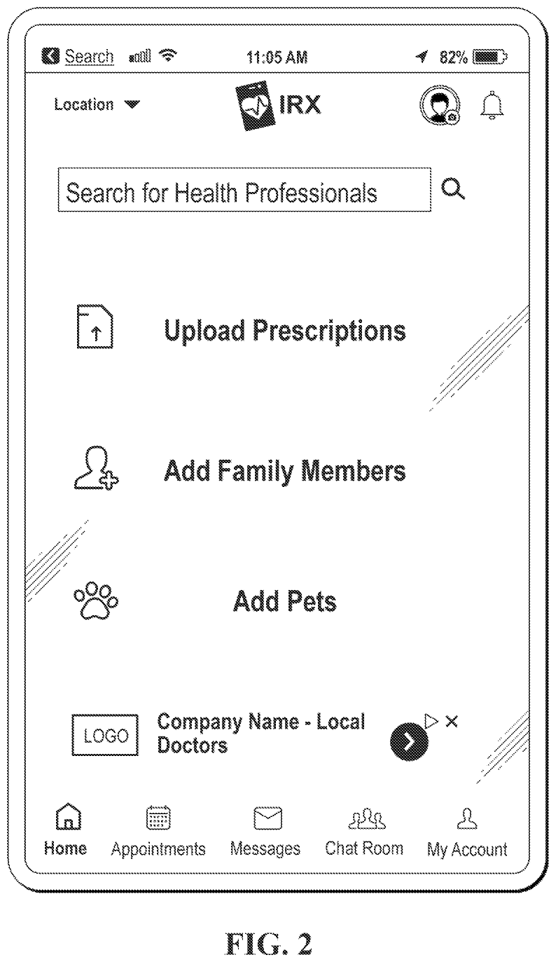

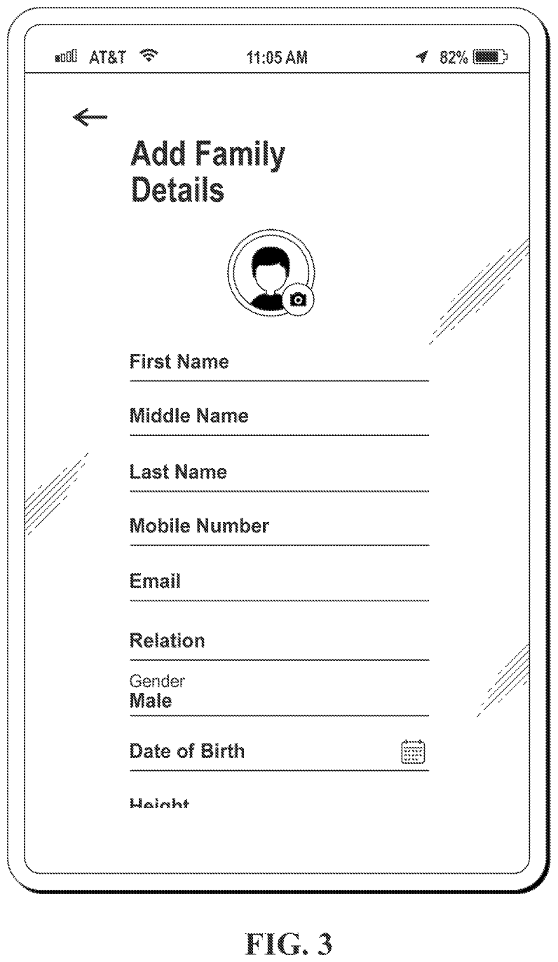

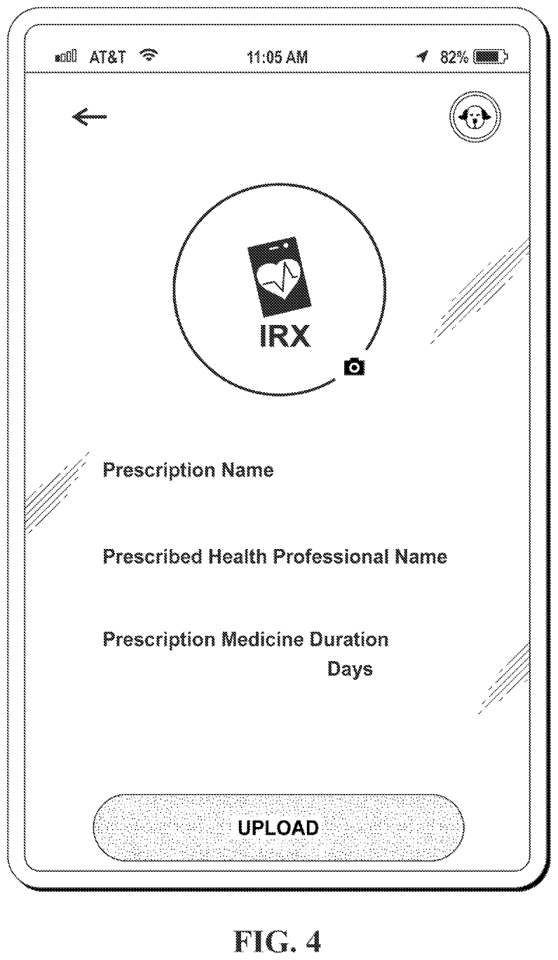

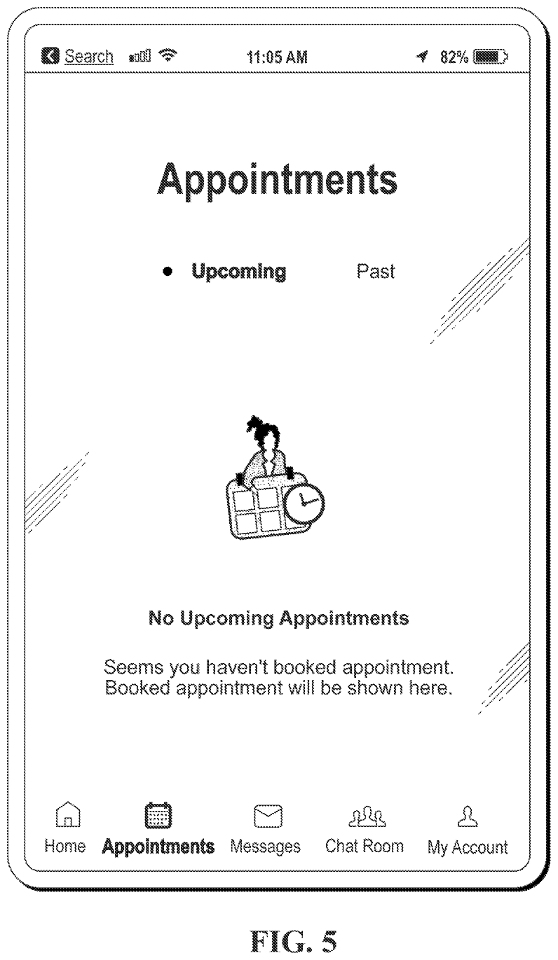

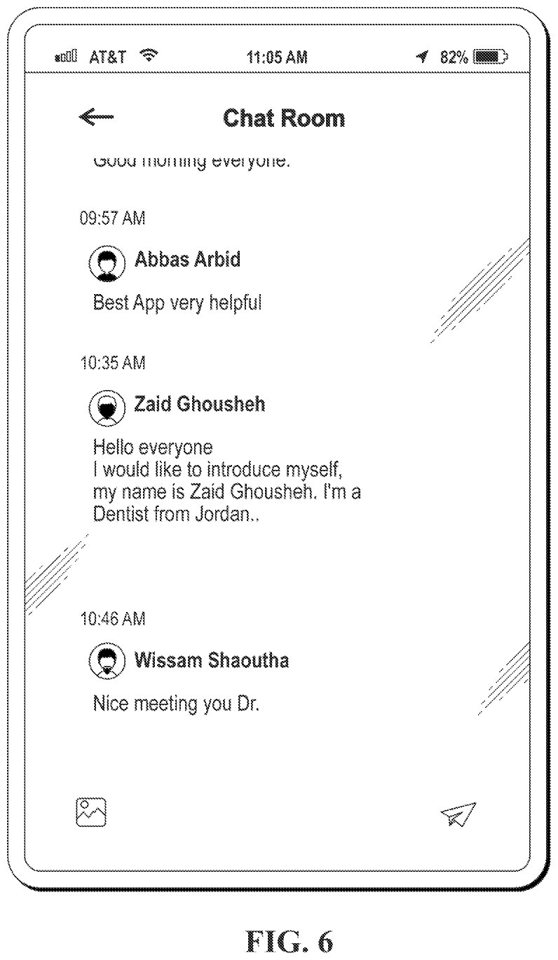

In most embodiments, the disclosed mobile application and web-based system (collectively, “system”) enables patients and healthcare professionals to connect and interact bi-directionally and wirelessly. The main steps are shown in FIG. 1. Representative user interfaces appear in FIGS. 2-8. Modes of communication include texting, voice communication, messaging, video calling and the opportunity to enter a “chat room” in which questions can be answered or cleared up and concerns addressed by communicating with a health professional. Appointments can be booked with health professionals nationwide. Video calling allows the healthcare professional to observe the patient, ask questions and make a diagnosis without the patient leaving his bed or home and without the doctor leaving his or her medical office. Such a feature would be of much to the bed-ridden patient. A chat room feature allows questions to be asked and answered quickly without jeopardizing confidentiality.

One result of using the app or web-based feature is to create a “medical wallet”. The medical wallet allows the patient to have in one place his or her (collectively, “their”) medical records, a list of medications and physicians for themselves or their loved ones, optionally including pets or other animals. Thus, the patient through the system has bi-directional access to healthcare professionals including physicians, dentists, pharmacies, laboratories and veterinarians, in any desired geographic area nationwide.

Preferably, embodiments of the system connect patients to doctors through a secure mobile application. Both groups may access the patient's medical profile, sports forms, medical lab reports and prescription information, among other data. In some embodiments, the patient may elect to share some or all his medical wallet with other health professionals. The application and web-based system are HIPAA-compliant and may have bank-rated encryption.

The system allows patients to have their medical information easily accessible by themselves and by the healthcare professional. In several embodiments, the system contains the necessary information for patients to locate doctors who honor their insurance. Further, it allows the patient to share his or her medical profile with others and fill out and share forms if desired. Optionally, patients can schedule appointments, securely chat with a doctor, receive prescriptions from their doctors and share the prescriptions with their pharmacy.

Several features of various embodiments of the system include:
A feature-rich, intuitive and user-friendly UI (user interface).
A fast connection between patients and healthcare professionals, which facilitates interactions between the two groups.
A vibrant, creative design and easy-to-use application.
A share button within the application which allows patients to share aspects of the system with others.

How The System Works

Consider FIG. 1. In a first step, the patient registers for a free account by downloading the app through the Apple App Store or Google Play. In one example, a patient may register to access one or more features of the system by using the Internet or other electronic communication means, such as a telephone, or facsimile. Typically, however, the patient uses a computer or mobile device to access an Internet registration web portal. This step may involve access via an Internet browser or a downloaded mobile app. The patient can sign up with their mobile number or social profile. The patient will create a personal profile and fill in the personal data necessary (i.e. photo id, height, weight, gender) and optionally provide relevant medical insurance information. All information in the app is saved to a secured server.

In some embodiments, the patient registration process may involve the patient providing the patient's contact information, his or her cardholder information, contact preferences and healthcare provider location preferences, including a specified location and/or a desired distance or radius, and the client's current location.

Healthcare professionals sign up via the web. To become a participating healthcare professional, the healthcare provider can register, thereby signifying his availability to be included among those healthcare providers with whom patients may communicate through the system. Again, registration can be facilitated via an Internet portal or otherwise, such as by telephone, letter, facsimile or other electronic communication means. The healthcare provider may designate identifying information, such as a national provider identifier (NPI).

In a second step, the patient chooses the health professionals within a geo-targeted area and can browse through the database and select the appropriate professional. Alternatively, the patient can click “friend request” to immediately connect with a healthcare professional. The healthcare professional may create and upload the medical records, lab results, imaging records, visitation and consultation notes as well as prescriptions to the app. A patient will have access to the files, but only health professionals will be able to access or alter them (with the permission of the patient).

In a third step, after connecting with the healthcare professional and participating in the visit in person, the results, including blood work, imaging results (a “medical wallet”) are stored in the patient profile. Optionally, the patient can rate the health professional on the app.

One or more central processing units lie in electronic communication with the patient's remote device and the healthcare provider. It will be appreciated that such computers may include processing devices configured for accessing and reading associated computer-readable media with information stored thereupon and/or computer-executable instructions for implementing the various features of the system.

Suitable software and/or hardware reside for receiving and transmitting data and/or computer-executable instructions over one or more communications links or networks. Suitable network devices and systems may include processors for processing data and instructions, together with controlling peripheral or internal components that are otherwise conventional. Memory devices may be provided that are configured to store data and/or instructions. Upon executing such instructions, at least some of the network devices comprise a computer.

Users

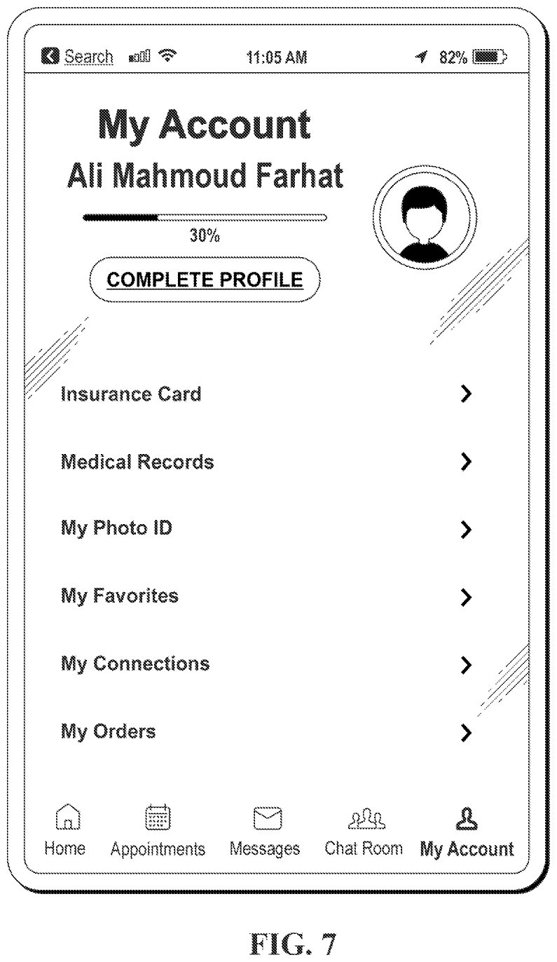

There are two primary users of this system. The first group of users include the patients who build their profile (see, e.g., FIG. 7) and visit the healthcare professionals. The second group of users are the healthcare professionals themselves.

Patients can view insurance information, their medical profile, lab results, sport forms, and prescriptions within a secure, mobile communications platform. As noted above, patients may also use the app to search for healthcare professionals in their geo-targeted area.

The patient may make a connection request. Then the healthcare professional can upload and delete files, view the patient's insurance information, medical history and any previous lab results or diagnosis. Through the mobile application, patients can schedule appointments, receive appointment confirmation notifications, share medical records or their medical profile, receive lab reports, and receive prescriptions and send the prescriptions to a pharmacy.

Through the mobile application, patients can upload their social security and insurance information to create a medical profile and securely chat with the doctor's office regarding their medical insurance or co-pay information, or schedule an appointment utilizing the office calendar through a dashboard-type feature of the system.

Healthcare professionals and patients are connected in a secure, user-friendly mobile application format. Optionally, video calling may be used to facilitate the patient-physician dialog without the patient needing to leave their bed or home. Using the platform, healthcare professionals can manage their schedule, accept or reschedule appointments, send and manage medical history and any previous lab results or diagnosis, send new patient forms, lab results, and prescriptions without the associated costs of additional office staff, phones, etc.

Further, the system may provide the ability to follow-up prior to a scheduled visit and a patient check-in post visit. As a connected healthcare professional, the application allows instant access to a patient's medical profile, including their insurance information to verify co-pay and claim amounts.

Optionally, a patient may search for a healthcare professional, limiting the search if desired by zip code or distance from the patient's location. In this way, the patient may for example search for nearby physicians, pharmacists, dental hygienists, dentists, nurses, surgeons, athletic trainers, exercise physiologists and surgical technologists.

In response to the patient's inquiry, for example, a listing of cardiologists may be presented on the patient's remote device, together with the relevant credentials of the cardiologist.

A healthcare professional can be included among those medical specialists with which the app may communicate by registering, preferably through a related website. In this way, the healthcare professional can ensure that his or her practice is among those, based on his specialty in a geographic area, to start connecting with patients.

The healthcare professional thus has an opportunity to stand out from competition in the same area of specialty. Further, in some embodiments, the app can be used pre-screen patients, validate insurance, and book more appointments. This drives a more efficient and profitable practice.

The application is expected to lower the costs of administrative services, increase patient demand, and assist the healthcare professionals in providing a higher level of personalized care. As more insurance companies accept virtual and online video chats, healthcare professionals can utilize a video calling feature to connect with their patients. Health care professionals can provide a higher level of care and cut down on administrative costs with the two-way secure chat and messaging function. Patients will not need to call the office to get hours, fax numbers, check up on their lab reports or prescription refills as such tasks can be securely done through the system. Once the visit is complete, healthcare professionals can securely share the post-visit report, at-home care documents, lab reports, prescriptions, or work-related letters via the website.

One aspect of the disclosure is a system such as an app that makes it easy for patients to interact with a healthcare provider using remote devices like smartphones or iPads or laptops or desktop computers (collectively, “remote devices”). Such devices preferably include a memory that stores textual and graphical information. Textual information may for example include details about blood work and other information typically housed in a medical record. Medical records may, for example, include doctor notes and records, lab results, imaging, and medications. Graphical images may for example include x-rays. Other medical test results can be downloaded on the system for future reference. A patient will have ready access to all results and prior medications taken before and after visiting a doctor.

The patient may permit other users, such as other family members to become involved in various aspects of app usage.

As used herein the term “healthcare provider” includes a medical doctor, a physical therapy practitioner, a pharmacist, or a veterinary doctor. A healthcare professional may practice medicine, surgery, dentistry, midwifery, pharmacy, psychology, nursing or allied health professions. See, en.wikipedia.org/wiki/Healthprofessional. As used herein “healthcare professional” also includes nurse practitioners, physician assistants, certified nurse specialists trained in a particular field such as E/R, pediatric or diabetic nursing, certified nurse midwives, certified registered nurse anesthetists and clinical social workers.

A central processor communicates with remote devices wirelessly. It will be appreciated that a patient's device or computer can include a mobile smart phone (e.g., an Android-based phone, a Microsoft-based mobile phone, or an Apple iPhone) or a similar communication device, such as a tablet computer. Patients may upload a prescription requirement to the central processor. Preferably, in one embodiment, an electronic prescription may be provided in an E-prescribing Electronic Data Interchange (EDI) format. Alternatively, in accordance with one embodiment of the invention, prescriptions can be received by the patient's remote device or an Internet website.

If desired, the app will remind patients of upcoming refills.

In this disclosure, some embodiments of the system make reference to a “patient”. In practice, the patient may be an individual who is in communication with a healthcare provider. In other examples, the patient may be someone who is authorized by or is acting on behalf of the patient. Such cases may include a person authorized to act on behalf of a minor or who is the patient. In other examples, a caregiver or spouse may be the one who is authorized to act on behalf of the patient.

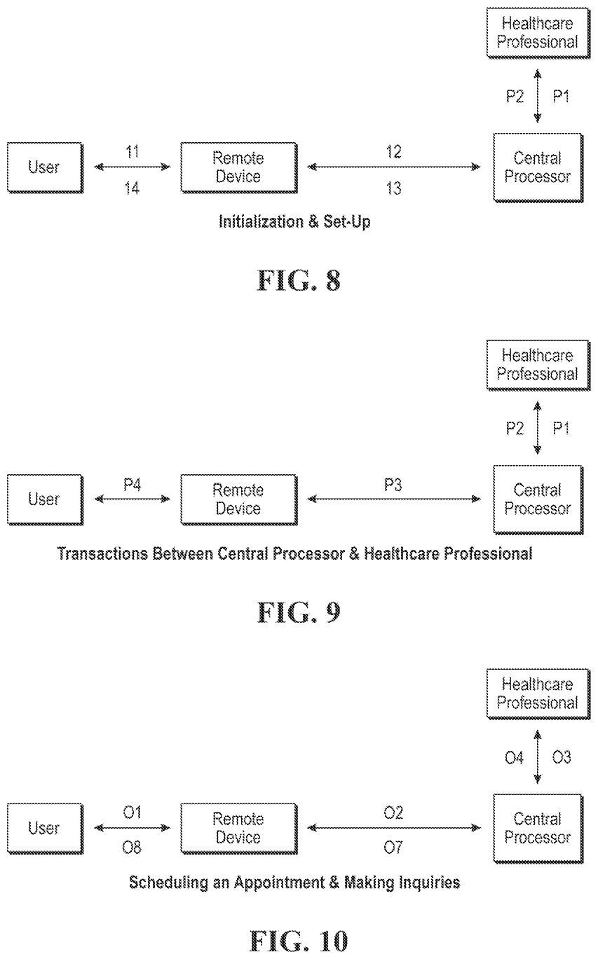

In practice, as shown in FIG. 8 a user will register with the system by creating an account at his remote device (transaction I1). To do this, a picture ID and insurance information for the primary user may be required. Details of other family members may be supplied to permit their enrollment. Account information is then uploaded to a central processor (transaction I2).

The central processor may house information about participating healthcare professionals or providers (Figures. 9-10).

Via the central processor, the user may schedule an appointment with a provider (FIG. 10, transactions O1, O2). The central processor will survey its database of participating providers (transactions O3, O4). That information is summarized and communicated to the user via his remote device (transactions O7, O8). Preferably, the information is presented in a sequence that lists providers by location. Optionally, the information could be sequenced by distance from the patient's location. Armed with such information, the user may then decide which provider is best suited to fulfill the medical need. He then schedules a visit via his remote device to the central processor, which in turn communicates the order to the selected provider (FIG. 10, transactions O3, O4).

Preferably the provider acknowledges receipt of the scheduling request (transactions O4, O7, O8) and communicates that fact to the user via the central processor together with a suggested appointment time(s).

In the embodiments described herein, the remote device may include a keypad, a memory and a processor. The remote device may be portable or be a desktop or laptop computer. The remote device may include a built-in router and be capable of wireless communication with a central processor. Users may access and control certain settings of the central processor via for example a hypertext markup language (“HTML”) 5 user interface using a web browser on the mobile device. Thus, since a web browser may open the user interface, the central processor settings may be implemented on any mobile device, regardless of the type, brand, or operating system of the device. No additional device specific application is required. The mobile device may then communicate directly with the central processor and apply the desired settings via user interaction at the mobile device. The user interface at the mobile device may be configured to save settings, profiles, prescriptions, etc., which may be easily recalled and applied.

FIGS. 8-10 illustrate for example one central processing unit, of which there may be one or more (collectively referred to herein as a “central processing unit”) in accordance with some embodiments. The central processing unit may have wireless communication capabilities, as described herein. The central processing unit may include an Ethernet™ connection, additional USB ports and a power source connection, and a cascade connector (e.g., cat-5 connector) to facilitate the cascading of multiple central processing units.

The central processing unit may also include an antenna and a light emitting diode (LED) 160, which may be configured to illuminate to indicate a connection with a wireless network. Although not shown, the Ethernet™ connection, reset switch, footswitch, additional USB ports and HDMI output may be arranged on a side panel of the central processing unit.

The central processing unit may include at least one handle and may be configured to be portable and easily moved from one location to the next. The central processing unit may also be rack-mountable.

The system may also include any number of mobile devices. Each mobile device may include a wireless transceiver (e.g., a BLUETOOTH module, a ZIGBEE transceiver, a Wi-Fi transceiver, an IrDA transceiver, an RFID transceiver, etc.) configured to communicate with the central processing unit and/or a remote server.

Each mobile device may include a device display screen configured to display information to a user and to receive commands from the user. The interfaces displayed via the display screen may be any one or a combination of visual displays such as light emitting diodes (LEDs), organic LED (OLED), Active-Matrix Organic Light-Emitting Diode (AMOLED), liquid crystal displays (LCDs), thin film diode (TFD), cathode ray tube (CRT), plasma, a capacitive or resistive touchscreen, etc.

As shown in FIGS. 8-10, each mobile device is configured to communicate with the central processing unit. Each mobile device may be capable of accessing an HTML5 webpage (i.e., the device may be HTML5 compatible). The mobile device may communicate directly with the central processing unit via a wireless network (not shown). Further, each mobile device may be any device capable of handling HTTP regardless of the device's platform (i.e., any device including iOS®, Android®, Windows®, Mac® OS, Linux®, etc., platforms). The central processing unit, as explained, may be capable of wireless communication without the use an external router. Thus, additional hardware and set-up thereof is not necessary to enable wireless communication with the central processing unit.

Each remote server or central processor may store and transmit updates (e.g., additional signal processing algorithms) to each mobile device.

As shown, the central processing unit may include a wireless access point and a web server configured to process requests via, by example, HTML, specifically HTML5. The wireless access point may facilitate a connection to the wireless network. Thus, commands may be sent from the mobile device directly to the central processing unit without the need for an external router. The web server may include Hyper Text Markup Language (HTML) 5, web sockets to open an interactive communication session between the user's browser and a server. By using a web socket-based application program interface (API), messages may be transmitted and received without having to poll an external server for a reply.

In use, once a user at the mobile device opens an app or a browser and enters the appropriate uniform resource identifier (URL), the user may enter his or her credentials (e.g., user name and password) or allow identification via a finger print. The mobile device employs such a user input to transmit a command over a socket connection to the web server of the central processing unit. The socket connection facilitates communication between the mobile device and the web server. Once this bi-directional communication is open, the central processing unit may receive commands from the mobile device. Concurrently, the web server may send feedback to the mobile device and the user interface at the mobile device may be updated accordingly.

The central processor may communicate with multiple mobile devices at a time. When multiple mobile devices are simultaneously transmitting commands to the central processor, each mobile device may, in real-time or near real-time, display the effects of the commands sent by another mobile device. For example, if a first user at a first one of the mobile devices transmits a scheduling request, a second user at a second one of the mobile devices may then see the request placed by the first user at the second mobile device. In addition to processing being recognized nearly simultaneously across multiple mobile devices, each mobile device operates independently of the other.

It will be appreciated that the central processing unit may include a processor and a one or more databases configured to perform instructions, commands and other routines in support of the processes described herein. For example, the central processor may be configured to execute signals generated by commands entered by a user at a remote device to generate signals(s) to provide an input to a healthcare provider. The processor may include a controller (not shown) and may include a dual-core processor (e.g., an ARM® processor) configured to interface with the web server and perform other signal processing if needed on-board the central processing unit.

A database may save historical transaction records created or placed by one or more users. These records may be user-specific and the database may maintain user profiles and settings associated therewith. Groups and sub-groups, including settings and security information therefor, may be maintained in the database. The database may receive updates from the mobile device, including software updates, as well as updated user information (including user profile updates and settings).

The mobile device may access and communicate with the central processing unit identification such as an internet protocol (IP) address of the central processing unit. Once the mobile device accesses the web server via the web socket, various user settings, presets, etc., may be adjusted in the HTML5 interface. Certain settings may be saved and recalled for later use. Additionally, certain security settings may be included via username/password combinations that are enterable at the HTML5 interface. In these settings, specific access may be given to certain login combinations. For example, a first user, or administrator, may have access to change credit card or other payment information and adjust, change, save, etc., any and all settings for all of the users that are operably connected to the central processing unit. In another example, another user may only have access to the settings as they relate to his or her order.

Not shown are other example screens for the user interface to be displayed via the web browser on the mobile device. The interfaces include various features configured to control the central processing unit in response to user inputs at the display screen of the mobile device. Shortcuts to various features and settings may be included throughout the interface to increase usability and provide a better user experience. The various interfaces may facilitate certain navigation and user gesture techniques to create a user-friendly interface system.

Because the central processing unit may be controlled from any number of devices, the central processing unit may continually send updated data back to the mobile devices. As a user navigates through the interfaces on the mobile device, the screens will be updated in real-time or near real-time. Upon receiving user input that is indicative of a change, a command may be transmitted via the wireless communication between the mobile device and the central processing unit indicating the change. The central processing unit, upon receiving the command, may apply the change and if appropriate transmit the updated data back to the mobile device so that the corresponding screens reflect the change. The updated data is also transmitted to any other mobile device currently communicating with the central processing unit.

In one example case (FIG. 10 transactions O1-O4, O7-O8), the central processing unit may receive a mobile device command from the mobile device. The mobile device command may include for example a scheduling request or a request for a particular prescription or refill. The mobile device command may be received from any number of mobile devices. For example, the mobile device command may be transmitted by a laptop and another command may be transmitted by a mobile phone.

In response, the central processing unit may transmit a confirmation to the mobile device. Thus, each time a command is entered at the mobile device, updated signals are transmitted back to the mobile device so that the interfaces thereon reflect the current state of the central processing unit. That is, the display screen may be continually updated. Furthermore, regardless of which mobile device transmitted the mobile device command, each mobile device in communication with the central processing unit may receive the updated data and therefore be configured to display the most up to date interfaces.

Such processes may proceed until the central processing unit is powered down, or until each of the mobile devices is no longer communicating with the central processing unit.

In one example, the mobile device transmits various mobile device commands and displays in real-time or near real-time the state of the central processing unit. The user input may include opening a web browser on the device and entering user credentials such as a user-name and password or finger print.

After the user is authenticated via the remote server or web server, the mobile device may transmit, via a wireless network, a request to the central processing unit. In response to the request, the mobile device may receive confirmation of the request from the central processor.

Accordingly, the disclosed system includes a central processing unit that has the ability to be controlled by any connected device via a standard web browser without the need for device-specific applications, and without operating system limitations. The central processing unit is simple and secure, allowing the user to easily handle his or her needs for management of his/her medical needs. The web-based mobile device-controlled system may allow users to control the central processing unit and its settings remotely over a wireless network.

Computing devices if present, such as the central processing unit, remote device, external server, remote server etc., generally include computer-executable instructions, that may be executable by one or more computing devices such as those listed. Computer-executable instructions may be compiled or interpreted from computer programs created using a variety of programming languages and/or technologies, including, without limitation, and either alone or in combination, Java™, C, C++, Visual Basic, Java Script, Perl, etc. In general, a processor (e.g., a microprocessor) receives instructions, e.g., from a memory, a computer-readable medium, etc., and executes these instructions, thereby performing one or more processes, including one or more of the processes described herein. Such instructions and other data may be stored and transmitted using a variety of computer-readable media.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

1. A non-transitory computer-readable medium tangibly embodying computer-executable instructions of a software program, the software program being executable by a processor of a computing device to provide operations, the operations comprising:
presenting, via a web browser on a mobile device, a display screen to receive user input to control a central processing unit that is operably coupled to at least one healthcare provider;
transmitting from the user to the healthcare provider, via a wireless network, a command from the mobile device, the command indicating a scheduling request or inquiry or both, the mobile device command being generated in response to user input at the display screen of the mobile device;
receiving by the central processing unit, via the wireless network and in response to the mobile device command, the scheduling request or inquiry or both; and
after the receiving step, presenting an updated display screen on the mobile device, the updated display screen reflecting the healthcare provider's response as a result of the mobile device command to present a real-time display of that response.

2. The non-transitory computer-readable medium of claim 1, further comprising:
receiving by the healthcare provider, via the wireless network, the order from the central processing unit.

3. The non-transitory computer-readable medium of claim 2, wherein the updated display screen includes indicia that signify confirmation of the response by the healthcare provider.

4. The non-transitory computer-readable medium of claim 3, wherein the user input includes a scheduling request or inquiry or both.

5. The medium of non-transitory computer-readable claim 4, further comprising presenting at least one screen specific to the central processing unit corresponding to the scheduling request or inquiry or both.

6. The non-transitory computer-readable medium of claim 3, wherein the updated display screen includes a confirmation of a command having been issued by the user.

7. The non-transitory computer-readable medium of claim 1, wherein the updated display screen is continually updated based on one or more signals from the central processing unit.

8. A healthcare services provider system for satisfying a request for healthcare services transmitted by a user via a remote device, comprising:
a mobile device including an interface configured to:
present a display screen to receive user input to control a central processing unit that is operably coupled to at least one healthcare provider;
transmit, via a wireless network, a command from the mobile device indicating a scheduling request or inquiry or both, the command being generated in response to user input;
receive, via the wireless network and in response to the command, a signal indicative of command status from the central processing unit; and
present an updated display screen, the updated display screen reflecting the healthcare provider's response as a result of the command to present a real-time display of the response.

9. A healthcare services provider system including an app that permits patients to interactively communicate with one or more healthcare services providers, the users deploying remote devices like smartphones, iPads, or desktop or laptop computers (collectively, “remote devices”), the system including:
one or more of remote devices that include a memory that stores textual and graphical information;
the textual information including details about such attributes as blood work, prescription needs and medical records;
the graphical information including one or more images such as x-rays and other medical test results that can be downloaded from a central processor to the remote device, thereby affording to the patient access to at least some previous results and prior medications taken before and after visiting a doctor.

10. The healthcare services provider system of claim 9, wherein the app further includes means for providing a patient with an option to permit other users, such as other family members to use the app.

11. The healthcare services provider system of claim 9, wherein the app further includes means for permitting the user to create a record stored on the remote device for a pet and populate the record with details of the pet's needs for medications.

12. The healthcare services provider system of claim 9, wherein the app reminds patients of upcoming prescription refills.

13. The healthcare services provider system of claim 9, wherein the app permits a user to create an account at a remote device using a picture ID and insurance information for a primary user, the account optionally including details of other family members for enrollment, the account information being uploaded to the central processor.

14. A method for providing healthcare services by a healthcare professional to a person who is or wishes to become a patient, comprising the steps of:
receiving, by one or more computers, an inquiry associated with a patient, wherein the inquiry relates to a scheduling request or an inquiry;
determining, by the one or more computers an identification of one or more participating healthcare professionals;
communicating that identification to the patient via a remote patient-controlled device;
sending, by the patient via the remote patient-controlled device, a request or inquiry to a selected healthcare provider via the one or more computers;
receiving, by a healthcare provider from the patient device, the request or inquiry;
sending, by the healthcare provider to the patient-controlled device a response to the request or inquiry.
